(19) United States
(12) Patent Application Publication
(10) Pub. No.: US 2019/0388060 A1
(43) Pub. Date: Dec. 26, 2019

(54) IMAGING SYSTEM AND METHOD WITH LIVE EXAMINATION COMPLETENESS MONITOR

(71) Applicant: General Electric Company, Schenectady, NY (US)

(72) Inventor: Svein Arne Aase, Trondheim (NO)

(21) Appl. No.: 16/015,454

(22) Filed: Jun. 22, 2018

(51) Int. Cl. A61B 8/00; G06T 7/00; G06T 11/00; A61B 8/08

(52) U.S. Cl. CPC A61B 8/463; G06T 7/0014; G06T 11/00; A61B 8/4444; A61B 8/4483; A61B 8/14; A61B 8/5207; A61B 8/543; G06T 2207/10132; G06T 2207/20084; A61B 8/0891; A61B 8/465

(57) ABSTRACT

An ultrasound imaging system and method accesses an imaging protocol for an ultrasound imaging session. The imaging protocol includes designated views that are to be obtained to complete the imaging protocol. Image data is acquired with an ultrasound imaging system, and artificial intelligence is used to identify a portion of the image data corresponding to at least one of the designated views of the imaging protocol. The identified portion of the image data corresponding to the designated view(s) of imaging protocol is automatically saved, and a graphical progress-of-completeness indicator of the imaging protocol is displayed that indicates that one or more of: the designated views of the imaging protocol that have been acquired and/or that one or more additional designated views of the imaging protocol have yet to be acquired.

### FIELD

The subject matter disclosed herein relates generally to imaging systems.

### BACKGROUND

Imaging systems generate image data representative of imaged bodies. Some imaging systems are live imaging systems that can generate and display images of the bodies while the image data continues to be obtained. Ultrasound imaging systems are one example of such live imaging systems. These types of imaging systems differ from other imaging systems that capture image data of a body that is subsequently displayed to an operator of the imaging system after an imaging session is completed (e.g., all sought-after image data of the body has been obtained).

During an imaging session, an operator of an imaging system may wish to obtain certain views of the body being imaged. For example, an operator using an ultrasound imaging system may wish to obtain an apical two chamber view of a person's heart, an apical four chamber view of the person's heart, and an apical long axis view of the person's heart to complete the imaging session. But, the operator may forget which views have been obtained, may forget which views have not yet been obtained, and/or become distracted by one view and begin taking other views that are not required to complete the imaging session (while not obtaining the views required to complete the imaging session). This may occur in situations where the operator is required to complete several different imaging protocols on a single person during a single imaging session. When the operator is required to concurrently obtain many different views under different parameters for multiple, different protocols, it can be difficult for the operator to maintain watch on the parameters and views that have been obtained. As a result, the imaging session may be terminated without all needed views of the person being obtained. This can require an additional imaging session to be performed, which can interrupt and delay imaging sessions of other persons.

### BRIEF DESCRIPTION

In one embodiment, an ultrasound imaging method includes accessing an imaging protocol for an ultrasound imaging session. The imaging protocol includes one or more designated views that are to be obtained to complete the imaging protocol. The designated views can refer to designated imaging views, designated acquisition views, or designated insonification views. The method also includes acquiring image data with an ultrasound imaging system. The image data includes a plurality of different obtained views from a plurality of different positions. The method also includes automatically identifying (with artificial intelligence) a portion of the image data corresponding to one of the one or more designated views of the imaging protocol, automatically storing (in a memory) the portion of the image data corresponding to the one of the one or more designated views of imaging protocol, and displaying (on a display device) a graphical progress-of-completeness indicator of the imaging protocol that indicates that one or more of: the one of the one or more designated views of the imaging protocol has been acquired or that one or more additional designated views of the imaging protocol is yet to be acquired.

In one embodiment, an ultrasound imaging system includes an ultrasound imaging probe configured to acquire image data during an ultrasound imaging session. The image data includes a plurality of different obtained views from a plurality of different positions. The imaging system also includes one or more processors configured to access an imaging protocol for the ultrasound imaging session. The imaging protocol includes one or more designated views that are to be obtained to complete the imaging protocol. The one or more processors also are configured to automatically identify a portion of the image data corresponding to one of the one or more designated views of the imaging protocol. The imaging system also includes a memory configured to automatically store the portion of the image data corresponding to the one of the one or more designated views of imaging protocol. The one or more processors are configured to direct a display device to display a graphical progress-of-completeness indicator of the imaging protocol that indicates that one or more of: the one of the one or more designated views of the imaging protocol has been acquired or that one or more additional designated views of the imaging protocol is yet to be acquired.

In one embodiment, an imaging method includes accessing an imaging protocol for an ultrasound imaging session. The imaging protocol includes one or more designated views that are to be obtained to complete the imaging protocol. The method also includes acquiring image data with an imaging system. The image data includes a plurality of different obtained views from a plurality of different positions. The method also includes automatically identifying (with artificial intelligence) a portion of the image data corresponding to one of the one or more designated views of the imaging protocol, automatically storing (in a memory) the portion of the image data corresponding to the one of the one or more designated views of imaging protocol, and displaying (on a display device) a graphical progress-of-completeness indicator of the imaging protocol and the portion of the image data that corresponds with the one or more designated views of the imaging protocol, the graphical progress-of-completeness indicator indicating that one or more of: the one of the one or more designated views of the imaging protocol has been acquired or that one or more additional designated views of the imaging protocol is yet to be acquired.

### BRIEF DESCRIPTION OF THE DRAWINGS

The inventive subject matter described herein will be better understood from reading the following description of non-limiting embodiments, with reference to the attached drawings, wherein below:

FIG. 1 is a schematic diagram of an ultrasound imaging system in accordance with an embodiment;

FIG. 2 illustrates a flowchart of a method for automatically capturing views of an imaged body required by an imaging protocol while image data of the body continues to be obtained;

FIG. 3 illustrates examples of graphical progress-of-completeness indicators shown on a user interface that is shown in FIG. 1;

FIG. 4 illustrates additional examples of graphical progress-of-completeness indicators shown on the user interface that is shown in FIG. 1; and

FIG. 5 illustrates additional examples of graphical progress-of-completeness indicators shown on the user interface that is shown in FIG. 1.

### DETAILED DESCRIPTION

The subject matter described herein relates to imaging systems and methods that access an imaging protocol that dictates which views of a body are to be obtained. The imaging protocol optionally dictates conditions in which the views are to be obtained, such as physiological parameters of the body being imaged and/or acquisition parameters (e.g., settings) of the imaging system. The imaging system is controlled to obtain image data, and the imaging system can automatically determine whether any portion of the image data obtained by the imaging system contains a view required by the imaging protocol. The imaging system optionally can determine if the conditions required by the protocol are met as well. The imaging system can automatically determine whether a required view is obtained without the operator of the imaging system having to recognize or identify the view in the image data. The imaging system can automatically save the portion of the image data that contains the required view in a memory, and can update a graphical progress-of-completion indicator that is shown on a user interface (e.g., an electronic display device). This indicator can represent which views required by the imaging protocol have been captured and/or which views required by the imaging protocol remain to be captured. This can assist in educating the operator of what additional views of the body are needed.

At least one technical effect of the subject matter described herein is the automatic identification and capture (e.g., saving in memory) portions of image data that contain views of a body that are required by an imaging protocol while the image data continues to be obtained. Another technical effect is the tracking and notification of which views required by the protocol have been obtained and/or which additional views required by the protocol remain to be obtained.

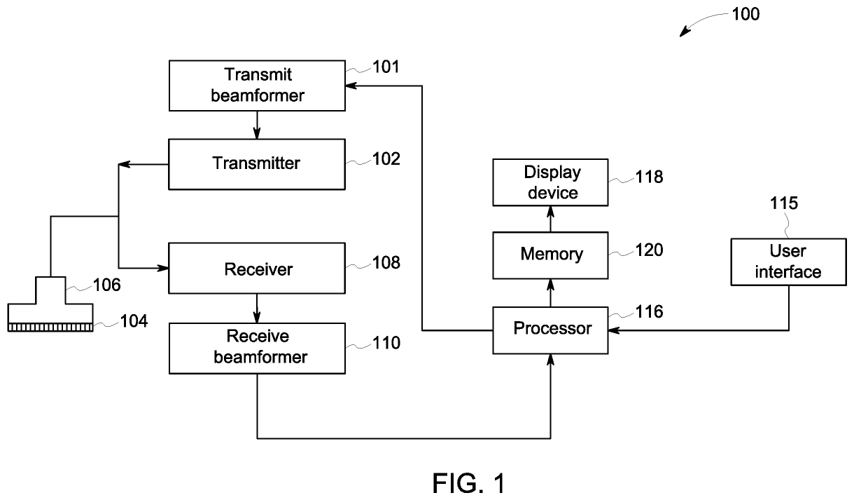

FIG. 1 is a schematic diagram of an ultrasound imaging system 100 in accordance with an embodiment. The ultrasound imaging system 100 includes a transmit beamformer 101 and a transmitter 102 that drive elements 104 within a probe 106 to emit pulsed ultrasonic signals into a body (not shown). According to an embodiment, the probe 106 may be a two-dimensional matrix array probe. However, another type of probe capable of acquiring four-dimensional ultrasound data may be used according to other embodiments. The four-dimensional ultrasound data can include ultrasound data such as multiple three-dimensional volumes acquired over a period of time. The four-dimensional ultrasound data can include information showing how a three-dimensional volume changes over time. Alternatively, a 1D array probe or a linear array probe may be used. Optionally, the system 100 may not acquire four-dimensional ultrasound data, but may obtain another type of imaging data, such as time motion ultrasound modes, plane video loops, or the like.

The pulsed ultrasonic signals are back-scattered from structures in the body, such as blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. The probe 106 may contain electronic circuitry to do all or part of the transmit and/or the receive beamforming. For example, all or part of the transmit beamformer 101, the transmitter 102, the receiver 108 and the receive beamformer 110 may be situated within the probe 106. Scanning may include acquiring data through the process of transmitting and receiving ultrasonic signals. Data generated by the probe 106 can include one or more datasets acquired with an ultrasound imaging system. A user interface 115 may be used to control operation of the ultrasound imaging system 100, including, to control the input of patient data, to change a scanning or display parameter, and the like. One example of a user interface 115 can be an electronic display device, such as a monitor, touchscreen, or the like. The user interface 115 optionally can include one or more input devices, such as keyboards, an electronic mouse, a speaker, etc.

The ultrasound imaging system 100 also includes one or more processors 116 that control the transmit beamformer 101, the transmitter 102, the receiver 108 and the receive beamformer 110. The processors 116 are in electronic communication with the probe 106 via one or more wired and/or wireless connections. The processors 116 may control the probe 106 to acquire data. The processors 116 control which of the elements 104 are active and the shape of a beam emitted from the probe 106. The processors 116 also are in electronic communication with a display device 118, and the processors 116 may process the data into images for display on the display device 118. The processors 116 may include one or more central processors (CPU) according to an embodiment. According to other embodiments, the processors 116 may include one or more other electronic components capable of carrying out processing functions, such as one or more digital signal processors, field-programmable gate arrays (FPGA), graphic boards, and/or integrated circuits. According to other embodiments, the processors 116 may include multiple electronic components capable of carrying out processing functions. For example, the processors 116 may include two or more electronic components selected from a list of electronic components including: one or more central processors, one or more digital signal processors, one or more field-programmable gate arrays, and/or one or more graphic boards. According to another embodiment, the processors 116 may also include a complex demodulator (not shown) that demodulates the radio frequency data and generates raw data. In another embodiment, the demodulation can be carried out earlier in the processing chain.

The processors 116 are adapted to perform one or more processing operations according to a plurality of selectable ultrasound modalities on the data. In one embodiment, the processors 116 may include one or more graphical processing units (GPUs), or may be communicatively coupled with one or more GPUs for performing analysis of the image data as described herein. The data may be processed in real-time during a scanning session as the echo signals are received, such as by processing the data without any intentional delay or processing the data while additional data is being acquired during the same imaging session of the same patient. For example, an embodiment may acquire images at a real-time rate of seven to twenty volumes per second. The real-time volume-rate may be dependent on the length of time needed to acquire each volume of data for display, however. Accordingly, when acquiring a relatively large volume of data, the real-time volume-rate may be slower. Some embodiments may have real-time volume-rates that are considerably faster than twenty volumes per second while other embodiments may have real-time volume-rates slower than seven volumes per second.

The data may be stored temporarily in a buffer (not shown) during a scanning session and processed in less than real-time in a live or off-line operation. Some embodiments of the inventive subject matter may include multiple processors (not shown) to handle the processing tasks that are handled by the processors 116 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a rate of, for example, ten to 200 hertz. Images generated from the data may be refreshed at a similar frame-rate. Other embodiments may acquire and display data at different rates. For example, some embodiments may acquire data at a rate of less than ten hertz or greater than 200 hertz depending on the size of the volume and the intended application.

A memory 120 is included for storing processed image data. In one embodiment, the memory 120 is of sufficient capacity to store at least several seconds or minutes worth of volumes of ultrasound data. The image data are stored in a manner to facilitate retrieval thereof according to its order or time of acquisition. The memory 120 may comprise any known data storage medium, such as one or more tangible and non-transitory computer-readable storage media (e.g., one or more computer hard drives, disk drives, universal serial bus drives, or the like).

Optionally, one or more embodiments of the inventive subject matter described herein may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters.

In various embodiments of the present invention, data may be processed by other or different mode-related modules by the processors 116 (e.g., B-mode, Color Doppler, M-mode, Color M-mode, spectral Doppler, Elastography, TVI, strain, strain rate, and the like) to form two- or three-dimensional image data. For example, one or more modules may generate B-mode, color Doppler, M-mode, color M-mode, spectral Doppler, Elastography, TVI, strain, strain rate and combinations thereof, and the like. The image beams and/or volumes are stored and timing information indicating a time at which the data was acquired in memory may be recorded. The modules may include, for example, a scan conversion module to perform scan conversion operations to convert the image volumes from beam space coordinates to display space coordinates. A video processor module may read the image volumes from a memory and displays an image in real time while a procedure is being carried out on a patient. A video processor module may store the images in an image memory, from which the images are read and displayed.

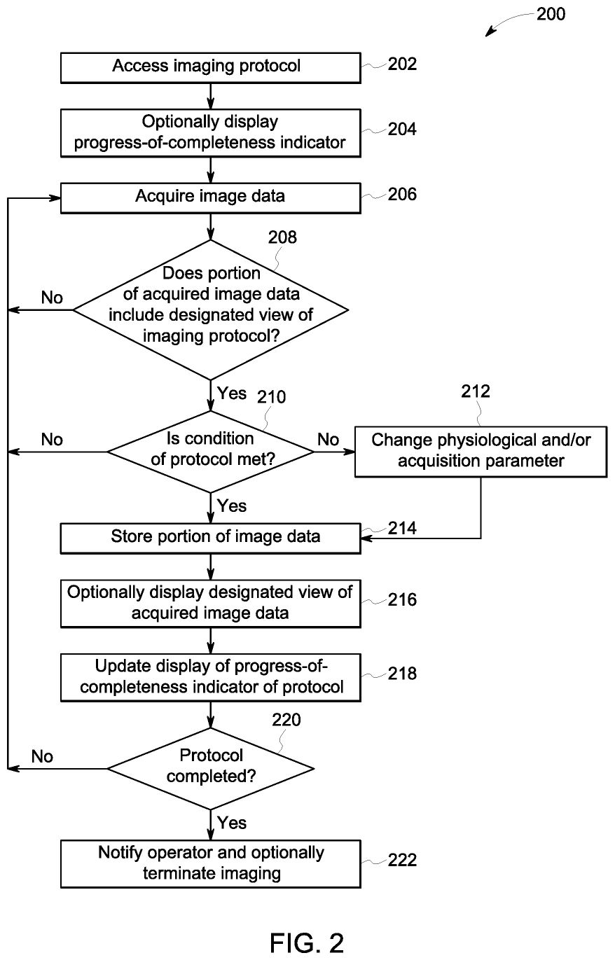

FIG. 2 illustrates a flowchart of a method 200 for automatically capturing views of an imaged body required by an imaging protocol while image data of the body continues to be obtained. The method 200 can represent operations performed by the processors 116 to automatically track which views required by an imaging protocol have been obtained and/or which views required by the protocol remain to be captured. At 202, an imaging protocol is accessed. The imaging protocol can be accessed by downloading or otherwise obtaining a copy of the protocol from the memory 120. Alternatively, the protocol can be provided from an operator of the imaging system 100 via the user interface 115.

The imaging protocol can be a list, table, or other memory structure that dictates or otherwise designates views of a body that are to be obtained to complete the imaging protocol. The views in the imaging protocol can designate orientations of images of an anatomical structure in a body being imaged. For example, an imaging protocol can require that an apical two chamber view, an apical four chamber view, an apical long axis view, and a view of a parasternal short axis view at the level of the papillary muscle (SAX), and the like, be obtained.

The imaging protocol can require that some or all the designated views be obtained in a designated order. For example, an imaging protocol can require that the apical two chamber view be obtained before the apical four chamber view, which is obtained before a subcostal view. Alternatively, the imaging protocol does not require that the views be obtained in a designated order. For example, the protocol can require that a view of the aortic valve, a subcostal view, and a 4CH view be obtained in any order or sequence.

The imaging protocol can dictate the views that are to be obtained to successfully complete an imaging session. An imaging session may begin when the imaging system 100 is activated and the probe 106 begins capturing image data of a body. The imaging session may continue so long as the body continues to be imaged by the probe 106, and can terminate when the probe 106 stops capturing image data of the body and/or the imaging system 100 is otherwise deactivated. For example, an imaging session may continue so long as the body continues to be imaged by the imaging system 100.

Optionally, an imaging protocol can dictate prerequisite conditions that must be met prior to or while one or more designated views of the protocol are acquired. These conditions can include one or more physiological parameters of the person being imaged. The physiological parameters can include a designated heart rate, a designated range of heart rates, a designated respiratory rate, a designated range of respiratory rates, and the like. For example, the imaging protocol may require that the heart rate of a person be at least one hundred beats per minute while a 4CH view is obtained, that the heart rate of the person be between eighty and ninety beats per minute while a subcostal view is obtained, that the respiratory rate of the person be no greater than twenty breaths per minute, or the like. These conditions can include one or more acquisition parameters of the imaging system 100. The acquisition parameters can include a frame rate, a range of frame rates, a resolution, an ultrasound line density, a range of ultrasound line densities, imaging width, imaging depth, ultrasound frequency, ultrasound pulse repetition frequency, ultrasound pulse length, power, or the like. The acquisition parameters may include an imaging mode, such as an ultrasound imaging mode. For example, an acquisition parameter may dictate that the image data be obtained using an ultrasound colorflow mode, an ultrasound pulsed wave Doppler mode, or the like.

The protocol can designate conditions across or among several views. For example, the protocol can require that two or more views (same or different views) be obtained while a physiological parameter (e.g., heart rate) does not vary by more than a designated amount (e.g., 20%). If any of the views are obtained while the physiological parameter varies by more than this amount, then the view does not satisfy the requirements of the protocol.

If a view required by the protocol is not obtained under or during the existence of a condition required by the protocol, then the view does not satisfy or meet the requirements of the protocol. But, if the view is obtained under or during the existence of the condition required by the protocol, then the view does satisfy or meet the requirements of the protocol.

At 204, the method 200 optionally includes displaying a progress-of-completeness indicator. This indicator informs the operator of the imaging system 100 of the completeness of the imaging protocol for an imaging session, and can be shown and updated while the image data is acquired by the imaging system 100 (and displayed to the operator). Thus, the indicator can provide a live completeness monitor for an examination of a person using the imaging system 100.

The indicator can be a graphical representation of how much of an imaging protocol is complete and/or how much of the imaging protocol remains to be completed. Optionally, the indicator can be a graphical representation of the views of the protocol that have been obtained, the views of the protocol that have not yet been obtained, the conditions required for capturing one or more views, or the like. The indicator can be shown on the user interface 115 along with the image data and/or views that are obtained by the probe 106. For example, the indicator can be shown alongside the image data as the image data is being acquired.

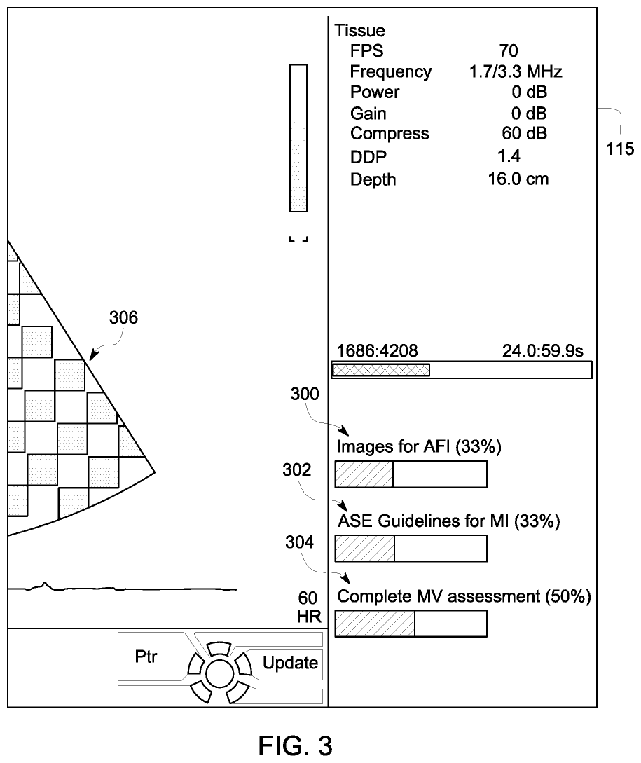

FIG. 3 illustrates examples of graphical progress-of-completeness indicators 300, 302, 304 shown on the user interface 115. The indicators 300, 302, 304 can be shown concurrently with and alongside a partial view 306 of image data. In the illustrated example, the indicator 300 represents how much of an imaging protocol that designates images associated with an automated functional imaging (AFI) protocol.

The indicator 302 represents how much of an imaging protocol that designates images used to examine or assess myocardial infarction (MI) according to guidelines established by the American Society of Echocardiography (ASE) has been captured. For example, one third of the images required by the guidelines established by the ASE to assess MI of a person have been obtained, and two thirds of the images required to by the guidelines remain to be obtained.

The indicator 304 represents how much of an imaging protocol that designates images used to examine or assess mitral valve (MV) prolapse according to guidelines established by the ASE has been captured. For example, half of the images required by the guidelines established by the ASE to assess MV prolapse of a person have been obtained, and one half of the images required to by the guidelines remain to be obtained.

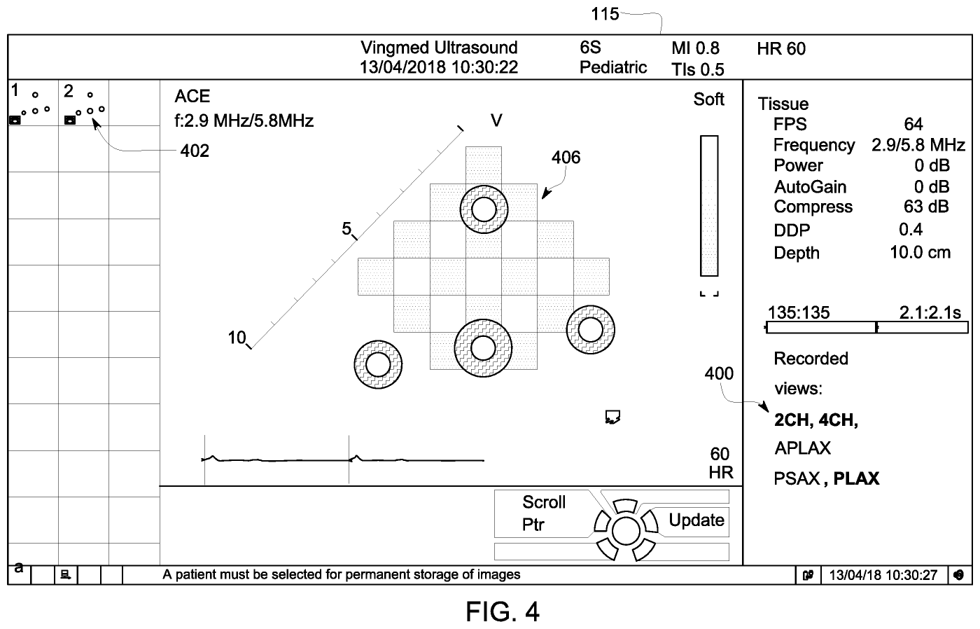

FIG. 4 illustrates additional examples of graphical progress-of-completeness indicators 400 shown on the user interface 115. The indicators 400 can be shown concurrently with and alongside a partial view 406 of image data. In the illustrated example, the indicator 400 is a textual list of views to obtain, such as 2CH, 4CH, an apical long axis (APLAX) view, a parasternal short axis view (PSAX), and a parasternal long axis (PLAX) view. Some of the views in the list of the indicator 400 may be shown in different colors, brightness, font, or the like, to represent which views have been obtained and which views have not yet been obtained. For example, the indicator 400 shows the terms 2CH, 4CH, and PLAX in different text (e.g., brighter or a different color of text) than the terms APLAX and PSAX, thereby indicating that the 2CH, 4CH, and PLAX views required by the imaging protocol have been obtained and the APLAX and PSAX views required by the imaging protocol have yet to be obtained. The interface 115 optionally can show smaller versions (e.g., thumbnails) of saved portions 402 of the image data that correspond to the required views of the imaging protocol.

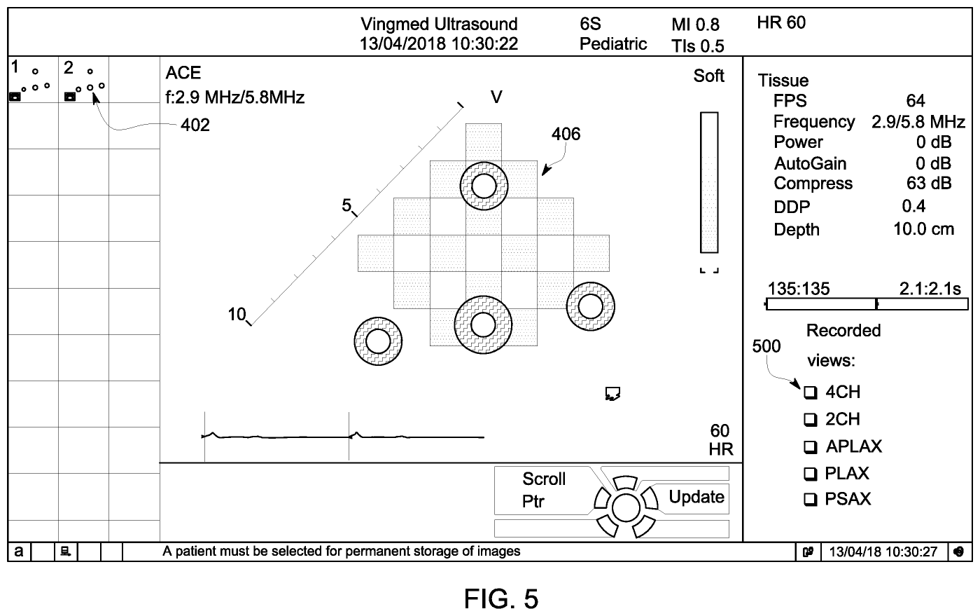

FIG. 5 illustrates additional examples of graphical progress-of-completeness indicators 500 shown on the user interface 115. The indicators 500 can be shown concurrently with and alongside the partial view 406 of image data. In the illustrated example, the indicator 500 is a textual checklist of views to obtain, such as 2CH, 4CH, APLAX, PSAX, and PLAX. The indicator 500 is a checklist with an annular graphical icon (e.g., a square, circle, or the like) next to each of the views in the checklist. An “X,” checkmark, or other symbol can be placed into the icon that corresponds with a view of the protocol that has been obtained. Those views of the protocol that have not yet been obtained can be shown without the symbol in the graphical icon.

Returning to the description of the flowchart of the method 200, at 206, image data is acquired. The operator of the imaging system 100 can move the probe 106 around the person being imaged to obtain image data of one or more anatomical structures (e.g., organs, vessels, bones, or the like) of the person being imaged. The image data can be presented on the user interface 115 while additional image data is being acquired during the same imaging session. For example, the user interface 115 can display a live view of the ultrasound image data as the image data is acquired and processed by the processors 116. The probe 106 of the imaging system 100 may be moved relative to the body being imaged so that the image data generated by the processors 116 of the imaging system 100 includes several different views of the imaged body from different positions. The different views can show different orientations of physiological structures in the body.

At 208, a determination is made as to whether any portion of the image data that is acquired includes a designated view required by the imaging protocol. If the image data includes a designated view of the imaging protocol, then at least part of the imaging protocol may have been completed. As a result, flow of the method 200 can proceed toward 210. But, if the image data acquired thus far does not include a designated view of the imaging protocol, then the imaging protocol has not yet been completed. As a result, flow of the method 200 can return toward 206. For example, additional image data can be acquired and examined in a loop-wise manner to determine when a designated view of the protocol has been obtained.

In one embodiment, the determination of whether a portion of the acquired image data includes a designated view of the imaging protocol is performed automatically by the processors 116. For example, the processors 116 may use artificial intelligence or other machine-based learning techniques to automatically determine if the image data represents a designated view. The artificial intelligence of the processors 116 can be embodied in one or more neural networks formed by at least some of the processors 116.

An artificial neural network formed by at least some of the processors 116 includes artificial neurons, or nodes, that receive input image data and perform operations (e.g., functions) on the image data, selectively passing the results of the operations onto other neurons. The neural network can operate to classify frames of the image data that is acquired. For example, the neural network can examine characteristics of a frame of image data and determine whether the frame belongs to one or more different classes of frames, such as apical two chamber views, apical four chamber views apical long axis views, or the like.

Alternatively, the neural network can identify objects in the frame of the image data, and determine what view the frame represents based on which identified objects appear in the frame. Weight values can be associated with each vector (described below) and neuron in the neural network, and these values constrain how input image data are related to outputs of the neurons. Weight values can be determined by an iterative flow of training image data through the neural network. For example, weight values are established during a training phase in which the neural network learns how to identify particular object classes by typical input image data characteristics of the objects in training or ground truth images.

A labeled training image can be image data where all or a substantial portion of the pixels or voxels forming the image data are associated with an object class. An object class is a type or category of an object appearing in the image data. For example, human tissue can be one object class, human bone can be another object class, a blood vessel can be another object class, and so on. A pixel or voxel can be labeled (e.g., associated) with probabilities that the pixel or voxel represents various object classes by a vector [a b c d], where the values of a, b, c, and d indicate the probability of the pixel or voxel representing each of different classes of objects or things. In a labeled training image, a pixel or voxel labeled as [1 0 0 0] can indicate that there is a 100% probability that the pixel or voxel represents at least a portion of an object of a first class (e.g., object class human tissue represented by probability a), a zero probability that the pixel or voxel represents at least a portion of an object of a different, second class (e.g., object class human bone represented by probability b), a zero probability that the pixel or voxel represents at least a portion of an object of a different, third class (e.g., object class blood vessel represented by probability c), and a zero probability that the pixel or voxel represents at least a portion of an object of a different, fourth class (e.g., object class representative of no portion of a body is represented by a probability d).

The artificial neurons in the neural network can examine individual pixels or voxels in input image data. The processors 116 can use linear classification to calculate scores for different categories of objects classes. These scores can indicate the probability that a pixel or voxel represents different classes. For example, the score for a pixel or voxel can be represented as one or more of the vectors described above. Each artificial neuron can apply a mathematical function, such as an activation function, to the same pixel or voxel, with the functions applied by different neurons impacting the functions applied by other neurons and different neurons applying different weights to different terms in the functions than one or more, or all other neurons. Application of the functions generates the classification scores for the pixels or voxels, which can be used to identify the objects in the input image data.

The neurons in the neural network examine the characteristics of the pixels or voxels, such as the intensities, colors, or the like, to determine the scores for the various pixels or voxels. The neural network examines the score vector of each pixel or voxel after the neural network has determined the score vectors for the pixels or voxels, and determines which object class has the highest probability for each pixel or voxel or which object class has a higher probability than one or more, or all, other object classes for each pixel or voxel. For example, a pixel or voxel having a score vector of [0.6 0.15 0.05 0.2] indicates that the neural network calculated a 60% probability that the pixel or voxel represents human tissue, a 15% probability that the pixel or voxel represents human bone, a 5% probability that the pixel or voxel represents a blood vessel, and a 20% probability that the first pixel or voxel represents nothing (e.g., not tissue, blood, or vessels). The processors 116 can determine that the pixel or voxel represents the object class having the greatest or largest of these probabilities. For example, the processors can determine that the pixel or voxel represents human tissue due to the 60% probability. This process can be repeated for several, or all, other pixels or voxels in the image data.

Once the neural network has identified likely object classes represented by the different pixels or voxels in the image data, the neural network can identify shapes formed by the pixels or voxels representing the same object class. These identified shapes can be compared with template shapes associated with different views of different anatomical structures (e.g., stored in the memory 120). If the identified shape of an object class (e.g., a blood vessel) more closely matches a shape template associated with a designated view of a blood vessel, then the processors 116 can determine that the image data shows the view of the blood vessel.

At 210, a determination is made as to whether a condition of the imaging protocol is met. As described above, the imaging protocol may require that one or more physiological parameters and/or acquisition parameters be met before a view of the imaged body is captured (e.g., saved in the memory 120). For example, the imaging protocol may require that the heart rate of the patient be within a designated range and that the frame rate of the imaging system 100 be at a designated rate. The heart rate of the patient or other physiological parameter may be measured by one or more sensors or input by an operator of the imaging system 100. The frame rate or other acquisition parameter can be determined by the processors 116 as the processors 116 control operation of the imaging system 100. If the imaging protocol includes one or more physiological conditions, acquisition conditions, or other conditions, and the conditions are not met, then a required view of the imaging protocol may not yet be obtained. For example, if the heart rate of the patient is not yet elevated to the designated heart rate range required by the imaging protocol, then a view of the imaging protocol may not yet be obtained. As a result, flow of the method 200 can proceed toward 212. But, if the imaging protocol includes one or more physiological conditions, acquisition conditions, or other conditions, and the conditions are met, then a required view of the imaging protocol may be captured and stored. For example, if the heart rate of the patient is elevated to within the designated heart rate range required by the imaging protocol, then a view of the imaging protocol may be obtained. As a result, flow of the method 200 can proceed toward 214.

At 212, the physiological and/or acquisition parameter is changed. For example, the physiological parameter of the person being imaged and/or the acquisition parameter of the imaging system 100 can be changed to be within the range or to be equal to the condition(s) required by the imaging protocol. With respect to physiological parameter conditions, this can involve the processors 116 instructing the person being imaged to increase (or decrease) their heart rate, such as by walking on a treadmill, sitting still, or the like. With respect to acquisition parameter conditions, this can involve the processors 116 changing one or more settings of the imaging system 100 to coincide with the condition of the imaging protocol. Once the condition or conditions of the protocol is or are met, flow of the method 200 can proceed toward 214.

At 214, a portion of the image data that corresponds with one or more of the designated views of the imaging protocol is stored. The processors 116 can automatically (e.g., without operator intervention) save a digital copy of the portion of the image data containing the view required by the imaging protocol in the memory 120. Alternatively, the processors 116 can direct the interface 115 to display a notification responsive to determining that a designated view of the imaging protocol has been obtained. The operator of the system 100 can then provide input that directs the processors 116 to save the designated view in the memory 120. Because the image data can be continuously obtained and/or shown to the operator while the probe 106 continues to obtain more views of the body, not all the image data may be saved in the memory 120. Instead, a subset or portion of the image data having the view required by the imaging protocol can be saved in the memory 120.

Optionally, the processors 116 may be configured to receive input from the operator that deletes or removes an obtained view of an imaging protocol from the memory 120. For example, although a designated view of the imaging protocol may be obtained, the operator may be displeased with the appearance or other features of the obtained view. The operator can provide input (e.g., via the interface 115) that indicates rejection of the obtained view and that directs the processors 116 to delete the obtained view. The processors 116 may then require that the operator capture the view again before the protocol is determined as being completed.

At 216, the method 200 optionally includes displaying the designated view of the imaging protocol from the acquired image data. For example, a graphical representation (such as a thumbnail view) of the saved portion 402 of the image data can be shown on the user interface 115, as described above.

At 218, the graphical progress-of-completeness indicator is updated or otherwise modified. The graphical progress-of-completeness indicator can be updated to show that at least one additional view required by the imaging protocol has been captured and saved. For example, one or more of the indicators 300, 302, 304 may be changed by the processors 116 to show that a greater percentage of the corresponding imaging protocol has been completed. The way one or more additional terms or words in the indicators 400 is displayed may be changed by the processors 116 to show that more views required by the imaging protocol have been obtained. One or more of the boxes or circles in the checklist of the indicator 500 may be checked by the processors 116 to show that more views required by the imaging protocol have been obtained.

At 220, a determination is made as to whether the imaging protocol is complete. The processor 116 can examine the views required by the imaging protocol and the portions of the image data that were automatically saved (e.g., at 214) to determine if all the views required by the imaging protocol have been obtained. If all views of the imaging protocol have been obtained, then the imaging protocol may be complete. As a result, flow of the method 200 can proceed toward 222. But, if one or more additional views of the imaging protocol need to be obtained, then the imaging protocol may not be complete. As a result, flow of the method 200 can return toward 210. For example, the method 200 can return to obtaining additional image data to determine if more views required by the imaging protocol are obtained.

Optionally, the processors 116 can generate a warning that is displayed on the interface 115 in the event that the operator of the system 100 attempts to end the imaging session before one or more imaging protocols are complete. For example, if one or more views of an imaging protocol have yet to be obtained but the operator attempts to close down or exit from the interface 115, the processors 116 can direct the interface 115 to generate a visual and/or audible warning to instruct the operator that one or more views of the imaging protocol still need to be obtained.

At 222, a notification of completion of the imaging protocol may be provided to the operator. For example, the processors 116 may change how the progress-of-completion indicator is displayed on the user interface 115 to indicate that the imaging protocol is complete. Optionally, the method 200 can terminate following 222. Alternatively, the method 200 can return to one or more other operations described above.

In one embodiment, an ultrasound imaging method includes accessing an imaging protocol for an ultrasound imaging session. The imaging protocol includes one or more designated views that are to be obtained to complete the imaging protocol. The method also includes acquiring image data with an ultrasound imaging system. The image data includes a plurality of different obtained views from a plurality of different positions. The method also includes automatically identifying (with artificial intelligence) a portion of the image data corresponding to one of the one or more designated views of the imaging protocol, automatically storing (in a memory) the portion of the image data corresponding to the one of the one or more designated views of imaging protocol, and displaying (on a display device) a graphical progress-of-completeness indicator of the imaging protocol that indicates that one or more of: the one of the one or more designated views of the imaging protocol has been acquired or that one or more additional designated views of the imaging protocol is yet to be acquired.

Optionally, the one or more designated views of the imaging protocol include one or more designated orientations of images of an anatomical structure in a body being imaged.

Optionally, the portion of the image data corresponding to the one of the one or more designated views is automatically identified using a neural network.

Optionally, the method also includes displaying (on the display device) the portion of the image data that corresponds with the one or more designated views of the imaging protocol on the display device.

Optionally, the graphical progress-of-completeness indicator and the portion of the image data that corresponds with the one or more designated views of the imaging protocol are concurrently displayed on the display device.

Optionally, the imaging protocol further comprises one or more prerequisite conditions that must be met while at least one or the one or more designated views are acquired.

Optionally, the one or more prerequisite conditions include a physiological parameter of a person being imaged.

Optionally, the physiological parameter is one or more of a designated heartrate, a designated heartrate range, a designated respiratory rate, or a designated respiratory rate range.

Optionally, the one or more prerequisite conditions include an acquisition parameter of the ultrasound imaging system.

Optionally, the acquisition parameter includes one or more of a designated frame rate, a designated frame rate range, a designated ultrasound line density, or a designated range of ultrasound line densities.

In one embodiment, an ultrasound imaging system includes an ultrasound imaging probe configured to acquire image data during an ultrasound imaging session. The image data includes a plurality of different obtained views from a plurality of different positions. The imaging system also includes one or more processors configured to access accessing an imaging protocol for the ultrasound imaging session. The imaging protocol includes one or more designated views that are to be obtained to complete the imaging protocol. The one or more processors also are configured to automatically identify a portion of the image data corresponding to one of the one or more designated views of the imaging protocol. The imaging system also includes a memory configured to automatically store the portion of the image data corresponding to the one of the one or more designated views of imaging protocol. The one or more processors are configured to direct a display device to display a graphical progress-of-completeness indicator of the imaging protocol that indicates that one or more of: the one of the one or more designated views of the imaging protocol has been acquired or that one or more additional designated views of the imaging protocol is yet to be acquired.

Optionally, the one or more designated views of the imaging protocol include one or more designated orientations of images of an anatomical structure in a body being imaged.

Optionally, the one or more processors also are configured to direct the display device to display the portion of the image data that corresponds with the one or more designated views of the imaging protocol on the display device.

Optionally, the imaging protocol further comprises one or more prerequisite conditions that must be met while at least one or the one or more designated views are acquired.

Optionally, the one or more prerequisite conditions include one or more of a physiological parameter of a person being imaged or an acquisition parameter of the imaging probe.

In one embodiment, an imaging method includes accessing an imaging protocol for an ultrasound imaging session. The imaging protocol includes one or more designated views that are to be obtained to complete the imaging protocol. The method also includes acquiring image data with an imaging system. The image data includes a plurality of different obtained views from a plurality of different positions. The method also includes automatically identifying (with artificial intelligence) a portion of the image data corresponding to one of the one or more designated views of the imaging protocol, automatically storing (in a memory) the portion of the image data corresponding to the one of the one or more designated views of imaging protocol, and displaying (on a display device) a graphical progress-of-completeness indicator of the imaging protocol and the portion of the image data that corresponds with the one or more designated views of the imaging protocol, the graphical progress-of-completeness indicator indicating that one or more of: the one of the one or more designated views of the imaging protocol has been acquired or that one or more additional designated views of the imaging protocol is yet to be acquired.

Optionally, the portion of the image data corresponding to the one of the one or more designated views is automatically identified using a neural network.

Optionally, the imaging protocol further comprises one or more prerequisite conditions that must be met while at least one or the one or more designated views are acquired.

Optionally, the one or more prerequisite conditions include a physiological parameter of a person being imaged.

Optionally, the one or more prerequisite conditions include an acquisition parameter of the ultrasound imaging system.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements that do not have that property.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An ultrasound imaging method comprising:
accessing an imaging protocol for an ultrasound imaging session, the imaging protocol including one or more designated views that are to be obtained to complete the imaging protocol;
acquiring image data with an ultrasound imaging system, where the image data comprises a plurality of different obtained views from a plurality of different positions;
automatically identifying, with artificial intelligence, a portion of the image data corresponding to one of the one or more designated views of the imaging protocol, wherein the portion of the image data is automatically identified as the one or more designated views of the imaging protocol in a sequence other than a designated sequence;
storing, in a memory, the portion of the image data corresponding to the one of the one or more designated views of imaging protocol; and
displaying, on a display device, a graphical progress-of-completeness indicator of the imaging protocol that indicates that one or more of: the one of the one or more designated views of the imaging protocol has been acquired or that one or more additional designated views of the imaging protocol is yet to be acquired.

2. The ultrasound imaging method of claim 1, wherein the one or more designated views of the imaging protocol include one or more designated orientations of images of an anatomical structure in a body being imaged.

3. The ultrasound imaging method of claim 1, wherein the portion of the image data corresponding to the one of the one or more designated views is automatically identified using a neural network.

4. The ultrasound imaging method of claim 1, further comprising displaying, on the display device, the portion of the image data that corresponds with the one or more designated views of the imaging protocol on the display device.

5. The ultrasound imaging method of claim 4, wherein the graphical progress-of-completeness indicator and the portion of the image data that corresponds with the one or more designated views of the imaging protocol are concurrently displayed on the display device.

6. The ultrasound imaging method of claim 1, wherein the imaging protocol further comprises one or more prerequisite conditions that must be met while at least one or the one or more designated views are acquired.

7. The ultrasound imaging method of claim 6, wherein the one or more prerequisite conditions include a physiological parameter of a person being imaged.

8. The ultrasound imaging method of claim 7, wherein the physiological parameter is one or more of a designated heartrate, a designated heartrate range, a designated respiratory rate, or a designated respiratory rate range.

9. The ultrasound imaging method of claim 6, wherein the one or more prerequisite conditions include an acquisition parameter of the ultrasound imaging system.

10. The ultrasound imaging method of claim 9, wherein the acquisition parameter includes one or more of a designated frame rate, a designated frame rate range, a designated ultrasound line density, or a designated range of ultrasound line densities.

11. The ultrasound imaging method of claim 1, further comprising:
determining that an operator of the imaging system is terminating the imaging session prior to completion of the imaging protocol; and
displaying, on the display device, a warning that informs the operator that one or more of the designated views of the imaging protocol have not been acquired.

12. The ultrasound imaging method of claim 1, further comprising:
determining whether an operator indicates rejection of the portion of the image data that was stored;
removing the portion of the image data from the memory responsive to determining that the operator indicates rejection of the portion of the image data; and
updating the graphical progress-of-completeness indicator to indicate that the designated view that corresponds with the portion of the image data that was removed from the memory still needs to be obtained.

13. An ultrasound imaging system comprising:
an ultrasound imaging probe configured to acquire image data during an ultrasound imaging session, the image data including a plurality of different obtained views from a plurality of different positions;
one or more processors configured to access accessing an imaging protocol for the ultrasound imaging session, the imaging protocol including one or more designated views that are to be obtained to complete the imaging protocol, the one or more processors configured to automatically identify a portion of the image data corresponding to one of the one or more designated views of the imaging protocol, the one or more processors configured to identify the portion of the image data as the one or more designated views of the imaging protocol in a sequence other than a designated sequence; and
a memory configured to automatically store the portion of the image data corresponding to the one of the one or more designated views of imaging protocol,
wherein the one or more processors are configured to direct a display device to display a graphical progress-of-completeness indicator of the imaging protocol that indicates that one or more of: the one of the one or more designated views of the imaging protocol has been acquired or that one or more additional designated views of the imaging protocol is yet to be acquired.

14. The ultrasound imaging system of claim 13, wherein the one or more designated views of the imaging protocol include one or more designated orientations of images of an anatomical structure in a body being imaged.

15. The ultrasound imaging system of claim 13, wherein the one or more processors also are configured to direct the display device to display the portion of the image data that corresponds with the one or more designated views of the imaging protocol on the display device.

16. The ultrasound imaging system of claim 13, wherein the imaging protocol further comprises one or more prerequisite conditions that must be met while at least one or the one or more designated views are acquired.

17. The ultrasound imaging system of claim 16, wherein the one or more prerequisite conditions include one or more of a physiological parameter of a person being imaged or an acquisition parameter of the imaging probe.

18. An imaging method comprising:
accessing an imaging protocol for an ultrasound imaging session, the imaging protocol including one or more designated views that are to be obtained to complete the imaging protocol;
acquiring image data with an imaging system, where the image data comprises a plurality of different obtained views from a plurality of different positions;
automatically identifying, with artificial intelligence, a portion of the image data corresponding to one of the one or more designated views of the imaging protocol, wherein the portion of the image data is automatically identified as the one or more designated views of the imaging protocol in a sequence other than a designated sequence;
automatically storing, in a memory, the portion of the image data corresponding to the one of the one or more designated views of imaging protocol; and
displaying, on a display device, a graphical progress-of-completeness indicator of the imaging protocol and the portion of the image data that corresponds with the one or more designated views of the imaging protocol, the graphical progress-of-completeness indicator indicating that one or more of: the one of the one or more designated views of the imaging protocol has been acquired or that one or more additional designated views of the imaging protocol is yet to be acquired.

19. The ultrasound imaging method of claim 18, wherein the portion of the image data corresponding to the one of the one or more designated views is automatically identified using a neural network.

20. The ultrasound imaging method of claim 18, wherein the imaging protocol further comprises one or more prerequisite conditions that must be met while at least one or the one or more designated views are acquired.

21. The ultrasound imaging method of claim 20, wherein the one or more prerequisite conditions include a physiological parameter of a person being imaged.

22. The ultrasound imaging method of claim 20, wherein the one or more prerequisite conditions include an acquisition parameter of the ultrasound imaging system.
